(19) United States
(12) Patent Application Publication
(10) Pub. No.: US 2026/0233995 A1
(43) Pub. Date: Aug. 13, 2026

(54) ANAEROBIC DIGESTER-BASED SYSTEM FOR RNG AND HYDROGEN PRODUCTION

(71) Applicant: Christian Tasser, Irvine, CA (US)

(72) Inventor: Christian Tasser, Irvine, CA (US)

(21) Appl. No.: 19/531,323

(22) Filed: Feb. 5, 2026

Related U.S. Application Data
(60) Provisional application No. 63/756,615, filed on Feb. 10, 2025.

(51) Int. Cl. C01B 3/58

(52) U.S. Cl. CPC C01B 3/586; C01B 2203/042; C01B 2203/0475; C01B 2203/062

(57) ABSTRACT

A method comprising first purifying a syngas mixture containing hydrogen to generate a hydrogen stream of higher purity than that in the syngas mixture and a waste gas stream then and then feeding the waste gas stream into an anaerobic digester to enable performance of a biomethanation process on the waste gas.

### RELATED APPLICATION

This application claims the benefit of and priority to U.S. Provisional Patent Application Ser. No. 63/756,615 filed Feb. 10, 2025, and entitled Anaerobic Digester-Based System for RNG and Hydrogen Production, the contents of which are incorporated by reference in its entirety herein.

### FIELD OF THE DISCLOSURE

The subject disclosure relates generally to waste-to-energy conversion systems.

### DESCRIPTION OF RELATED ART

Systems for processing the gas output of an anaerobic digester have been proposed in the past.

### SUMMARY

In an illustrative embodiment, a syngas mixture containing hydrogen is first purified to generate a hydrogen stream of higher purity than that in the syngas mixture and a waste gas stream. The waste gas stream is then fed into an anaerobic digester to enable performance of a biomethanation process on the waste gas.

According to another aspect of the disclosure, a tubular distribution array is provided within the anaerobic digester to distribute the waste gas over the bed of the digester. In one illustrative embodiment, the array may comprise a generally rectangular planar array of tubes or pipes which are perforated along a length of each tube to obtain a fine bubble distribution of the waste gas over the surface of the bed of the digester.

According to another aspect of the disclosure, an apparatus is provided configured to first purify a syngas containing hydrogen to generate two streams: a hydrogen stream of higher purity than that in the syngas and a waste gas stream.

The apparatus is further configured to input the waste gas stream into an anaerobic digester to enable performance of a biomethanation process on the waste gas.

### DESCRIPTION OF THE DRAWINGS

FIG. 1 is a block diagram of an integrated waste-to-energy conversion system employing an anaerobic digester according to an illustrative embodiment;

FIG. 2 is the left side of a detailed schematic diagram illustrating apparatus for processing the output of the anaerobic digester of FIG. 1 according to an illustrative embodiment;

FIG. 3 is the right side of the diagram of FIG. 2;

FIG. 4 is a front view of an illustrative SMR reactor according to an illustrative embodiment;

FIG. 5 is a side perspective view of a WGS reactor according to an illustrative embodiment;

FIG. 6 is a front perspective view of a boiler according to an illustrative embodiment;

FIG. 7 is a partial exploded perspective view illustrating internal aspects of the boiler of FIG. 6;

FIG. 8 is a table describing various legends used in FIGS. 2 and 3; and

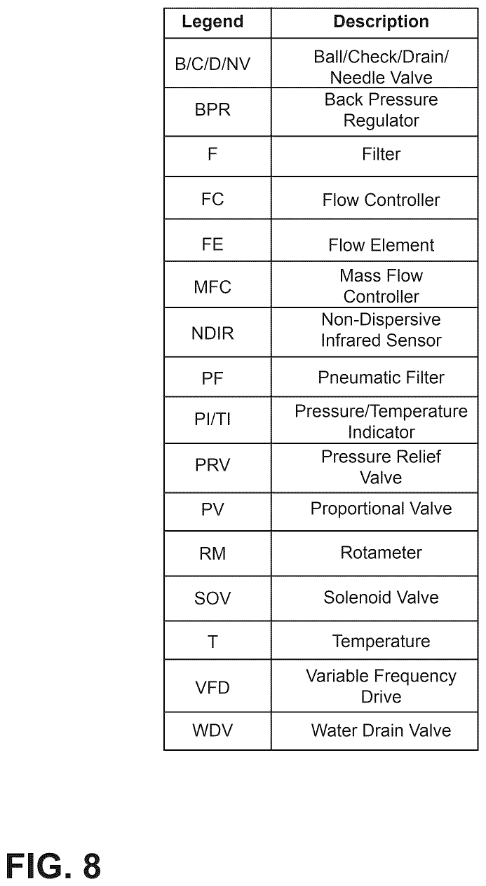

FIG. 9 is an illustrative embodiment for distributing waste gas within an anaerobic digester.

### DETAILED DESCRIPTION

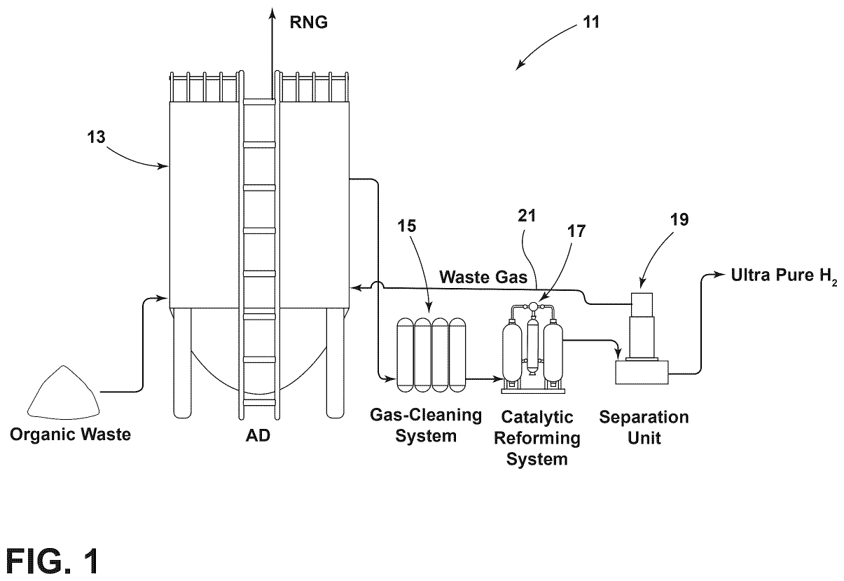

An illustrative embodiment of an integrated waste-to-energy conversion system 11 is illustrated in FIG. 1. This system 11 includes an anaerobic digester 13, a gas cleaning system 15, a catalytic reforming system (“CRS”) 17, and a hydrogen separation unit 19.

The anaerobic digester 13 is designed to handle various types of organic waste, including, for example, municipal solid waste (MSW), agricultural residues, and industrial food waste. As known in the art, under anaerobic conditions, microorganisms break down the organic matter and convert gas-phase CO2 into biomethane (biogas). The digester 13 of the illustrative embodiment is also designed to accommodate further biomethanation of a waste stream 21 generated by and received from the separation unit 19. In another embodiment, the digester 13 may be augmented by a separate anaerobic digester to further enhance the rate of biomethanation of the waste stream 21.

The raw biogas generated by the digester 13 is passed through the gas-cleaning system 15 comprising multiple units to remove trace impurities from the biogas to ensure that it is suitable for further processing in the CRS 17.

The hydrogen-rich gas mixture exiting the CRS 17 is then passed through the adsorption-based separation unit 19 that produces a high-purity hydrogen product and a separate hydrogen-lean waste stream 21 containing the carbon compounds in syngas. The waste stream 21 is fed into the anaerobic digester 13 for a biomethanation step to take place.

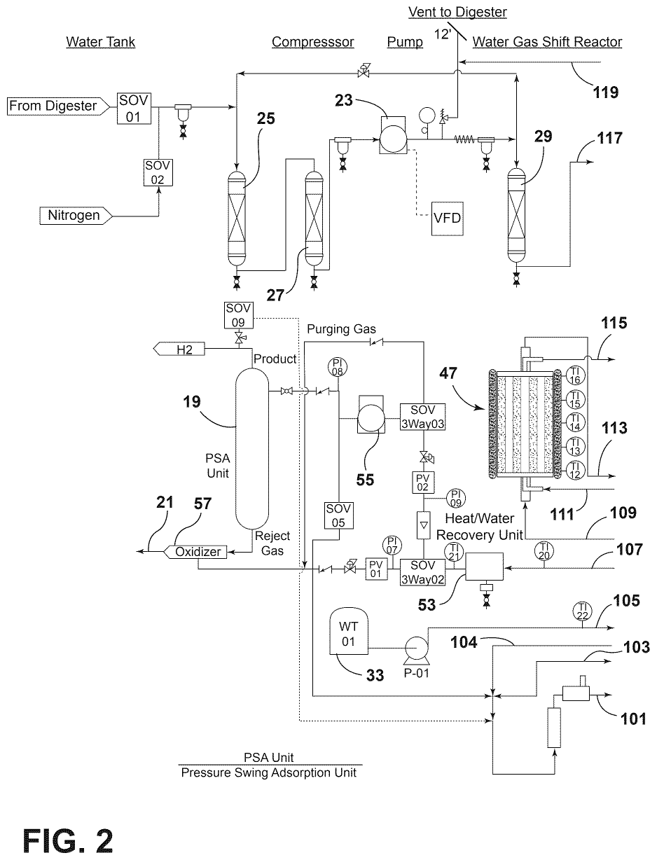

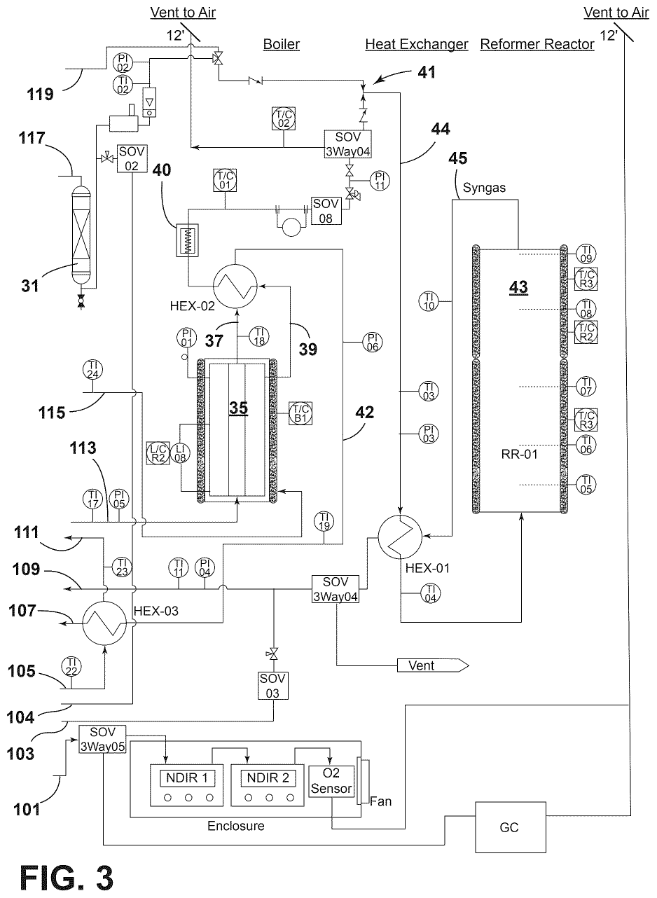

FIGS. 2 and 3 illustrate in more detail a system configured according to FIG. 1. FIG. 2 is the left-hand portion of the system, while FIG. 3 is the right-hand portion. The interconnections between the two Figures are numbered down the right-hand side of FIG. 2 and the left-hand side of FIG. 3. For example, line 101 at the bottom of FIG. 2 connects with line 101 on FIG. 3, line 103 on FIG. 2 connects with line 103 on FIG. 3, etc.

The system of FIGS. 2 and 3 includes a compressor 23 for moving the biogas input from the digester 13. In the illustrative embodiment, the gas cleaning system comprises adsorbing columns 25, 27 upstream of the compressor 23 and adsorbing columns 29, 31 downstream of the compressor 23. The upstream adsorbing columns 25, 27 may use DARCO activated carbon (AC) to remove hydrogen sulfide and silica gel (SiG) to remove water vapor before the gas is fed to the compressor 23, while the downstream columns 29, 31 may also include AC and SiG to remove remaining contaminants and water vapor after the gas has cooled.

Other gas cleaning systems may be used in alternate embodiments. The nitrogen input through SOV 02 is to purge the system at start-up and to insure there is no air left in the system.

The system of FIGS. 2 and 3 further includes a water tank 33 and a boiler 35 for generating steam containing an oxygen compound, which in the illustrative embodiment is water. The steam output 39 from the boiler 35 is passed through a heat exchanger HEX 02 and a coil 40 to a combiner 41 where it is combined with the biogas stream exiting the downstream adsorbing column 31. The coil 40 is heated using heat tapes to superheat the steam to a higher temperature. In the illustrative embodiment, the combiner 41 is a multiport valve but could be other gas mixing apparatus in other embodiments. In an illustrative embodiment, the output gas stream 44 from the combiner 41 may be: CH4: 3.28 mol/min, CO2: 2.18 mol/min, and Steam: 8.19 mol/min, but may be of other compositions in other embodiments.

The combined gas stream 44 containing methane, carbon dioxide, and steam is then passed through a heat exchanger HEX 01 and into a reformer reactor system comprising two reactors in series: (i) an electrified steam methane reforming reactor (SMR) reactor 43 containing a nickel-based supported catalyst, where the biogas/steam mixture reacts at high temperatures (>750 degrees C.) to produce a syngas exit stream mixture 45 containing H2, CO2 and carbon monoxide (CO) together with a minor CH4 content; (ii) a second reactor 47, which is a water gas shift (WSG) reactor containing an iron-based catalyst where the exit stream from the SMR reactor 43 further reacts at high temperatures (>300 degrees C.) to convert the CO in the exit stream from SMR reactor 43 into additional H2 and CO2. In the illustrative embodiment, the composition of the syngas exit stream 45 from the SMR reactor 43 may be: CO: 2.61 mol/min, H2: 8.75 mol/min, CH 4: 0.44 mol/min, CO2: 2.41 mol/min, Steam: 5.12 mol/min, but may be of different compositions in other embodiments.

The syngas stream 45 then passes through the heat exchanger HEX 01 and via line 109 to the water gas shift (WGS) reactor 47 to lower the CO content and increase the hydrogen content. The hot gas output 113 of the WGS reactor 47 is then passed via line 113 through the boiler 35 to heat the water inside the boiler 35 and exits the boiler 35 via line 37. In the illustrative embodiment, the composition of the hot gas output in line 113 may be: CO: 0.69 mol/min, H2: 10.67 mol/min, CH4: 0.44 mol/min, CO2: 4.33 mol/min, Steam: 3.20 mol/min, but may be of other compositions in other embodiments.

The gas mixture output of the boiler 35 in line 37 is then passed through the heat exchanger HEX 03 and over line 42 to the adsorption-based separation unit 19. In the illustrative embodiment, the composition of the gas flowing in line 42 may be: CO: 0.69 mol/min, H2: 10.67 mol/min, CH4: 0.44 mol/min, CO2: 4.33 mol/min, Steam: 3.20 mol/min, but may differ in different embodiments.

The gas in line 42 (line 107 in FIG. 2) is routed through the heat recovery unit 53 and directed by a first 3-way SOV and a subsequent second 3-way SOV to the compressor 55 and then into the PSA 19. Alternately, the first 3-way valve can be switched to direct a purging gas in line 107 through the oxidizer 57 and to the digester. The second 3-way SOV which feeds the compressor 55 can also be switched to direct purging gas down the vertical line ending in an arrowhead and into the oxidizer 57. The purging gas may be nitrogen and is employed to clean unwanted or hazardous gases from the system. Two sources of purging gas are provided for back-up redundancy.

In the illustrative embodiment, the adsorption-based separation unit 19 is a pressure swing adsorption unit (“PSA”). Prior to input to the PSA, the gas stream 42 passes through a heat recovery unit 53 and a second compressor 55. In an illustrative embodiment, the H2 output from the PSA is 99% pure and is produced at a rate of 25 kg per day.

Water is pumped from the water tank 33 (FIG. 2) to supply the feedwater for the boiler 35 of FIG. 3. The water from the water tank 33 first enters the heat exchanger HEX 03 via line 105, then exits that heat exchanger and is input via line 111 into the WGS reactor 47 in order to cool the reactor 47. The water then exits the WGS reactor 47 and enters the boiler 35 via line 115.

With respect to the heat exchangers, HEX 01 functions to heat the feed to the SMR system using hot gas coming out of the SMR 43, HEX 02 functions to heat steam using hot gas coming out of the boiler, and HEX 03 functions to heat the water using product gas. Depending on conditions, the water might be heated to, for example, 90 degrees C.

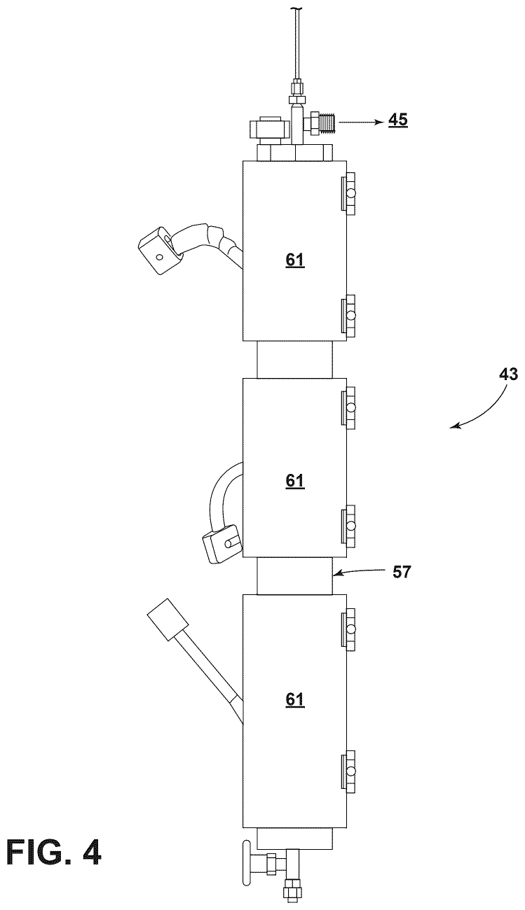

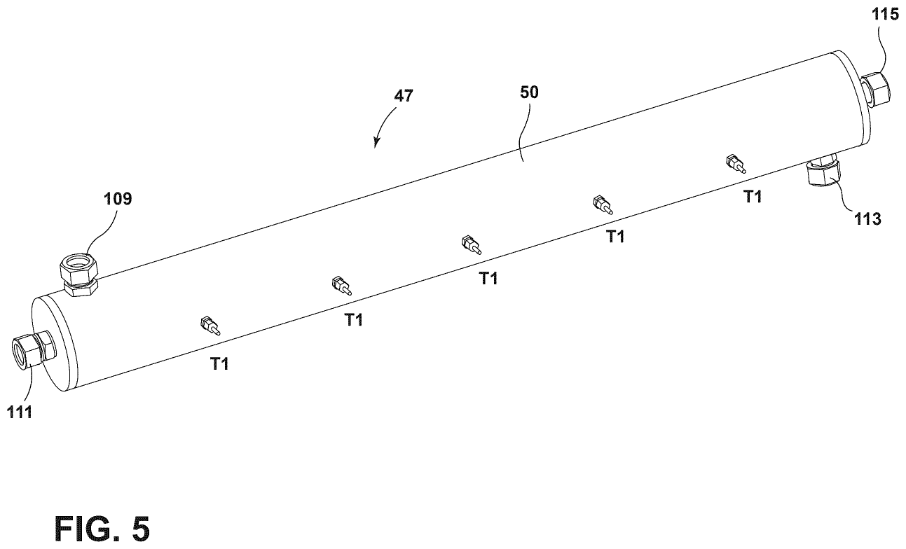

As shown in FIGS. 4 and 5, in the illustrative embodiment, the SMR reactor 43 may comprise a three-inch diameter stainless steel tube 57 with electrical heaters 61 around it, and the water gas shift (WGS) reactor 47 may be a four-inch diameter stainless steel tube 50 with a one-inch diameter tube inside it through which water passes as a cooling agent and includes temperature indicators T1 disposed along its length. In the illustrative embodiment, the WGS reactor 47 is designed to maintain high conversion with low pressure drop, and the cooling system is designed to maintain a constant temperature at around 350-400 degrees centigrade.

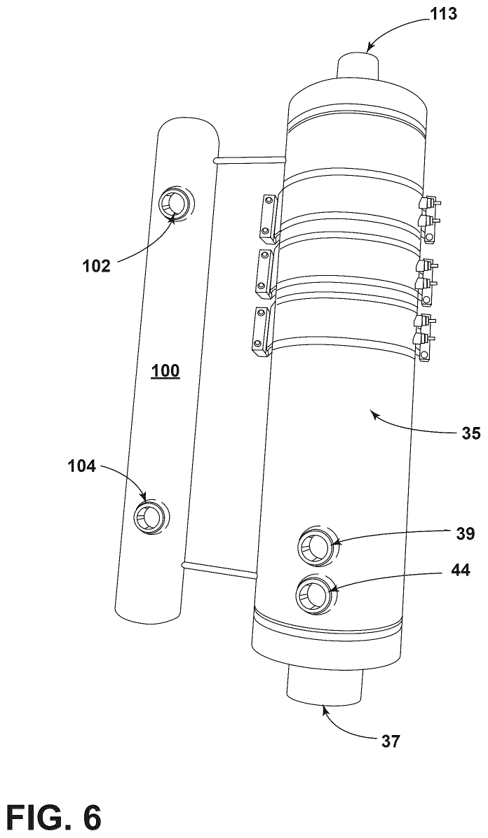

As illustrated in FIG. 6, the boiler 35 includes a central cylindrical stainless steel boiling chamber with a gas input from line 113, a gas output to line 37, and a steam output to line 39. The steam input 115 is on the opposite side of the cylinder 35. A connector opening 40 accommodates a pressure relief valve. The smaller cylinder 100 includes openings 102, 104 to connect a glass level indicator. In FIG. 6, component 100 depicts the level indicator made out of glass with openings 102 and 104 for liquid exchange to the level indicator.

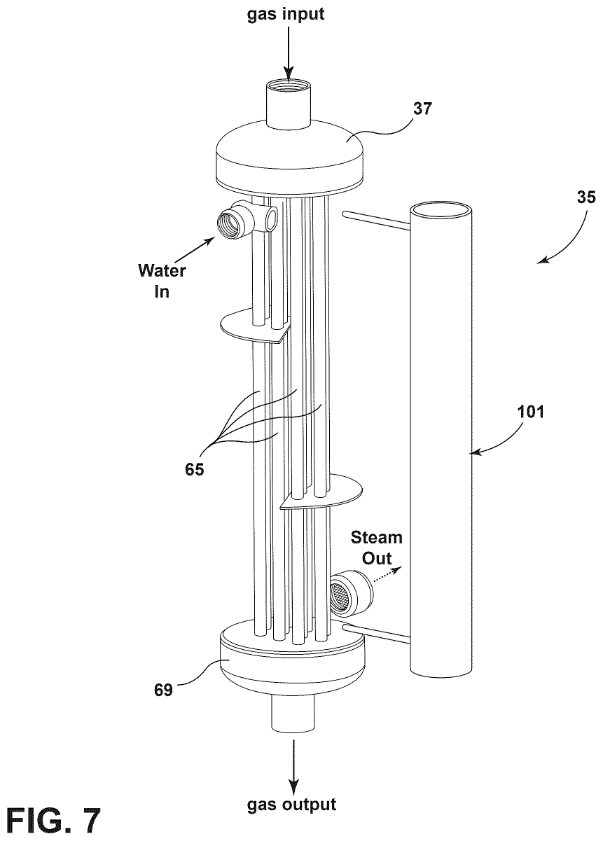

As shown in FIG. 7, internally, the boiler 35 includes a number of smaller gas transfer tubes 65 disposed between end caps 67, 69. The gas stream entering the boiler 35 is transferred via these tubes 69 to the output end cap 69 and heats the surrounding water introduced by the input line 115 to create the steam output from the boiler 35 via line 39.

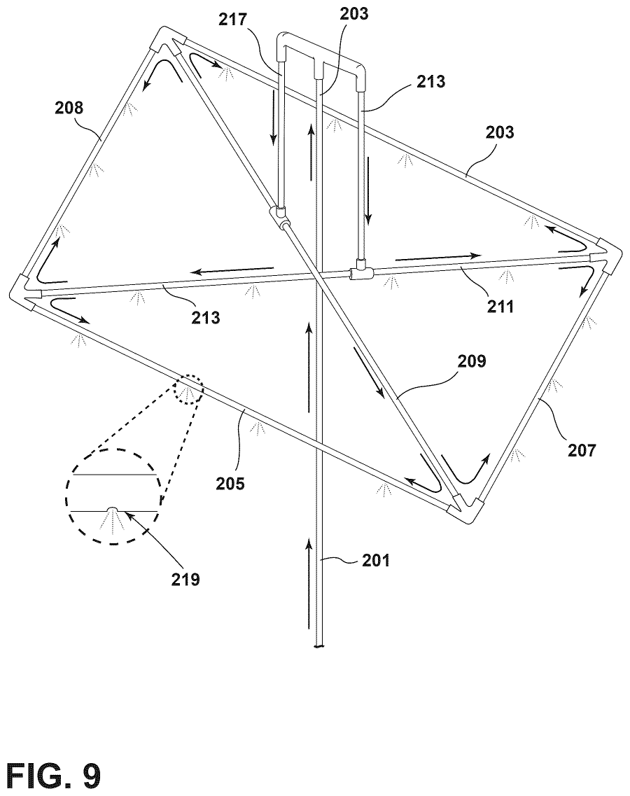

FIG. 9 is an illustrative embodiment of structure internal to the anaerobic digester to distribute the incoming waste gas. The waste gas, which comprises some hydrogen but mostly CO2, exits the PSA and passes through an oxidizer 57 to line 21, which feeds it to the digester 13. The oxidizer 57 can be used in case of emergency to burn the waste gas. The incoming waste gas is then input via a feed tube 201 to the distribution array of FIG. 9 positioned at the bottom of a tall digester tank.

The distribution array comprises a generally horizontal rectangular planar array of tubes or pipes 205, 207, 209, 211, 213, which are perforated, for example, every two inches along each tube, to obtain a fine bubble distribution of the waste gas at the bottom of the tank and over the entire surface of the bed of the digester.

The input waste gas proceeds as indicated by the arrows through a horizontal input tube 201, up vertical tube 213 and then down vertical tubes 215, 217 into the horizontal array, allowing for high mass transfer. The hydrogen and CO2 in the waste gas are absorbed by the microorganisms in the digestate liquid and converted to biomethane. If desired, the waste gas input to the digester 13 can be augmented in some embodiments, for example, by addition of supplemental hydrogen or CO2 employing, for example in some embodiments, a second input tube 219.

In one embodiment, the PSA may be a Xebec H3300 system available from Ivys Adsorption Inc., Blainville Quebec, Canada, but could be of other configurations in other embodiments. The WGS unit 47 may employ a commercially available catalyst such as Shandong Dengzhuo Chemical Co., Ltd (DZC-F96), and the SMR reactor 43 may also employ a commercially available catalyst such as Clariant Reformermax 330.

Thus, in operation, an anaerobic digester is used to convert organic waste into biogas, followed by the processing of the biogas in an integrated electrically-heated, waste heat recuperating, catalytic reformer system (CRS) to produce ultrapure hydrogen (H2) together with a secondary hydrogen-lean stream 21. This secondary stream undergoes further on-site biomethanation to produce renewable natural gas (RNG). Illustrative embodiments thus provide a versatile and efficient conversion of organic waste into high-valued energy products, namely RNG and H2, through the integration of anaerobic digestion with catalytic biogas reforming.

Other optional equipment which may be included in various embodiments include an electricity generator such as a solar and/or wind power generation system, supplemented with an energy storage system, an electrolysis unit for further enhancing bio methanation rates, if so desired, and/or a biogas upgrading system to produce food-quality CO2 or industrial-quality CO2 for sale.

Those skilled in the art will appreciate that various adaptations and modifications of the just described illustrative embodiments can be configured without departing from the scope and spirit of the invention. Therefore, it is to be understood that, within the scope of the appended claims, the invention may be practiced other than as specifically described herein.

What is claimed is:

1. A method comprising:
purifying a syngas mixture containing hydrogen to generate a hydrogen stream of higher purity than that in the syngas mixture and a waste gas stream; and
feeding the waste gas stream into an anaerobic digester to enable performance of a biomethanation process on the waste gas.

2. The method of claim 1 wherein the syngas comprises hydrogen and carbon monoxide.
